SEQUENCE LISTING (1 sequence)

SEQ ID NO: 1 - protein, 10 aa, Artificial Sequence
  Consensus amino acid sequence of hundreds of peptides purified from D. radiodurans
DEHGTAVMLK

(19) United States
(12) Patent Application Publication
(10) Pub. No.: US 2014/0314810 A1
(43) Pub. Date: Oct. 23, 2014

(54) Compositions Containing Purine and Pyrimidine Nucleosides, Peptides, and Manganese and Their Uses

(71) Applicant: Henry M. Jackson Foundation for the Advancement of Military Medicine, Inc., Bethesda, MD (US)

(72) Inventors: Michael Daly, Washington, DC (US); Elena K Gaidamakova, Gaithersburg, MD (US); Ian A. Myles, Kensington, MD (US); Patricia A. Valdez, Kensington, MD (US); Sandip K. Datta, Bethesda, MD (US)

(73) Assignee: United States Department of Health and Human Services, Bethesda, MD (US)

(21) Appl. No.: 14/355,063

(22) PCT Filed: Nov. 1, 2012
(86) PCT No.: PCT/US2012/062998

§ 371 Date: Apr. 29, 2014

Related U.S. Application Data
(60) Provisional application No. 61/554,136, filed on Nov. 1, 2011.

(51) Int. Cl. A61K 39/193; A61K 39/085

(52) U.S. Cl. CPC A61K 39/193; A61K 39/085; A61K 2039/522
USPC 424/218.1; 424/243.1; 435/173.3; 435/173.1

(57) ABSTRACT

The invention provides methods of producing vaccines directed against methicillin-resistant Staphylococcus aureus (MRSA) or Venezuelan equine encephalitis virus (VEEV), with the methods comprising culturing, harvesting and/or suspending the MRSA or VEEV in the presence of a radiation-protective composition and irradiating the MRSA with a dose of radiation sufficient to render the MRSA or VEEV replication-deficient and/or non-infective. The radiation-protective compositions used in the methods of the present invention comprise at least one nucleoside, at least one antioxidant and at least one small peptide.

### GOVERNMENT SUPPORT

The present invention arose in part from research funded by grant FA9559-07-1-0128 from the Air Force Office of Scientific Research. The Government has certain rights in the invention.

### RELATED APPLICATIONS

N/A

### BACKGROUND OF THE INVENTION

1. Field of Invention

The invention provides methods of producing vaccines directed against methicillin-resistant Staphylococcus aureus (MRSA) or Venezuelan equine encephalitis virus (VEEV) with the methods comprising culturing, harvesting and/or suspending the MRSA or VEEV in the presence of a radiation-protective composition and irradiating the MRSA or VEEV with a dose of radiation sufficient to render the MRSA or VEEV replication-deficient. The radiation-protective compositions used in the methods of the present invention comprise at least one decapeptide in a mixture of manganese-phosphate or manganese-bicarbonate buffer.

2. Background of the Invention

Vaccine preparation normally requires expensive molecular characterization of epitopes and immunogens to develop recombinant vaccines. Whole organism vaccines are generally preferred over these recombinantly produced vaccines as these recombinant vaccines are costly, time consuming and often times not very immunogenic. Live attenuated vaccines can be effective in producing a strong immune response, but these vaccines can be unsafe as live pathogens are administered to subjects. To date, whole organism vaccine preparations using ionizing radiation to kill the bacteria and/or viruses have generally not been successful. The levels of radiation required to render viruses and/or bacteria safe, sterile and replication-deficient generally destroy the antigenic determinants, thus rendering the vaccines non-immunogenic and non-protective.

Similarly, most cells, whether eukaryotes, prokaryotes or from mammals (e.g. humans) are also not radiation resistant. In addition, most proteins are not radiation-resistant. As such, exposure to radiation is quite damaging to protein structure and/or function and the functions they catalyze. For example, ionizing radiation has been shown to induce (cause) cancer in many different species of animals and in almost all parts of the human body.

For example, superoxide can build up in cells during irradiation because superoxide does not readily cross membranes. Although superoxide does not react with DNA, superoxide will damage and inactivate enzymes with exposed 2Fe-2S or 4Fe-4S clusters, releasing Fe(II) and also damage certain exposed amino acids such as, but not limited to, Sistine. The problem with iron in a cell, when it is unbound and “free”, is that it causes Fenton reactions in the presence of hydrogen peroxide, generating hydroxyl radicals. Therefore, conditions which liberate bound Fe(II) are extremely dangerous, not only because of the generation of hydroxyl radicals, but because the loss of Fe from Fe-dependent enzymes leads to the failure of the biochemical pathways within which they operate.

The extremely radiation-resistant family Deinococcaceae is comprised of greater than twenty distinct species that can survive acute exposures to ionizing radiation (IR) (10 kGy), ultraviolet light (UV) (1 kJ/m²), and desiccation (years); and can grow under chronic IR (60 Gy/hour). In particular, Deinococcus radiodurans is an extremely ionizing radiation (IR) resistant bacterium that can survive exposures to gamma-radiation that exceed by a factor of one thousand the doses which are cytotoxic and lethal to mammalian cells

For extremely resistant bacteria such as D. radiodurans, survival following high-doses of IR has been attributed to protection of proteins from oxidation during irradiation, with the result that enzymic repair systems survive and function with far greater efficiency during recovery than in sensitive bacteria, where cellular proteins are highly susceptible to carbonylation and other forms or protein oxidation. In a report published in Science magazine (Daly et al. (2004), Accumulation of Mn(II) in Deinococcus radiodurans facilitates gamma-radiation resistance, Science 306: 925-1084), intracellular manganese(II) was implicated in facilitating radiation resistance by protecting proteins, but not DNA, during exposure to ionizing radiation; and in a second report published in PLoS Biology (Daly et al. (2007) Protein oxidation implicated as the primary determinant of bacterial radioresistance, PLoS Biology 5(4) e92), radiation resistance was positively correlated to protein protection during irradiation, mediated by a non-enzymic mechanism.

The inventors have studied the radio-resistance of D. radiodurans and prepared ultra-purified, protein free-cell extracts that exhibit radioprotective properties. The invention is based on the discovery of radioprotective components of D. radiodurans cell free extract and artificial compositions containing such components that confer resistance to ionizing radiation.

In particular, there is no known vaccination against methicillin-resistance Staphylococcus aureus (MRSA). MRSA is responsible for the majority of skin and soft tissue infections in humans. Indeed, MRSA strains present major problems related to cutaneous and soft tissue infection due to the high incidence of infection and the seemingly constant emergence of antibiotic-resistant strains. Invasive MRSA presents a true health hazard as invasive MRSA accounts for over 18,000 deaths per year in the United States alone, which is more than the number of deaths associated with HIV/AIDS, influenza and hepatitis combined.

There is thus an urgent need for a vaccine against MRSA. The vaccine against MRSA should be highly immunogenic, cost-effective and relatively quick to produce.

### SUMMARY OF THE INVENTION

The invention provides methods of producing vaccines directed against methicillin-resistant Staphylococcus aureus (MRSA) or Venezuelan equine encephalitis virus (VEEV), with the methods comprising culturing, harvesting and/or suspending the MRSA or VEEV in the presence of a radiation-protective composition and irradiating the MRSA or VEEV with a dose of radiation sufficient to render the MRSA or VEEV replication-deficient. The radiation-protective compositions used in vaccine preparation methods of the present invention comprise at least one decapeptide in a manganese-containing buffer.

### BRIEF DESCRIPTION OF THE DRAWINGS

FIG. 1 depicts the uncoupling of genome damage and killing from epitope destruction in viruses irradiated with Mn-DP-Pi. (a) Bacteriophage lambda survival. For batches of 10⁹ phage, complete inactivation (loss of all pfu) occurred at 5 kGy in Pi buffer, and 25 kGy in Mn-DP-Pi. 0 on y-axis corresponds to 100% survival. (b) Bacteriophage λ DNA damage assessed by Southern blotting using a ³²P-labeled λ DNA probe. (c) λ protein damage assessed by SDS-polyacrylamide gel electrophoresis and visualized by Coomassie staining. (d) Protection of antibody-binding epitopes of λ phage assessed by Western blotting using duplicate gels of the one shown in panel c. I, Antibodies raised against non-irradiated λ phage; II, Antibodies raised against λ phage exposed to 40 kGy in Mn-DP-Pi: Mn, 1 mM MnCl₂; DP, 3 mM decapeptide (DP; H-Asp-Glu-His-Gly-Thr-Ala-Val-Met-Leu-Lys-OH) (SEQ ID NO:1); Pi, 25 mM potassium phosphate buffer (pH 7.4). pfu, plaque forming units; EC, purified E. coli proteins (1.25 μg); M, protein size standards. Viability assays for phage irradiated in Pi or Mn-DP-Pi were in triplicate, with standard deviations shown.

FIG. 2 depicts VEEV (strain 3526) virus infectivity and epitope protection in the presence or absence of Mn-DP-Pi. (a) Infectivity following irradiation assessed in vitro by the cytopathic effect (CPE) assay after 5 successive passages in Vero cells. Live cells are stained by crystal violet. (b) Protection of antibody-binding epitopes assessed by Western blotting using the monoclonal antibody 13D4-1, which binds the E3 domain of the PE2 glycoprotein of V3526. (+), V3526 irradiated in Mn-DP-Pi. (+), V3526 irradiated in Pi buffer alone. λ phage head protein (H) and tail protein (T). MC, no virus, medium control. Other abbreviations as in FIG. 1.

FIG. 3 depicts Mn-DP-Pi protecting staphylococcal epitopes from radiation damage but not increasing survival. (a) Viability of Staphylococcus aureus (MRSA USA300 strain) after exposure to ionizing radiation in the absence or presence of Mn-DP-Pi. (b) Binding of anti-MRSA IgG in immune serum (1:5000 dilution) to plates coated with MRSA that had been irradiated in the absence or presence of Mn-DP-Pi. (c) Binding of anti-MRSA IgG in immune serum (1:5000 dilution) to protein A-deficient Wood 46 strain of S. aureus or MRSA USA300 that had been irradiated with 25 kGy in the absence or presence of Mn-DP-Pi. (d) Anti-S. aureus IgG titers in serum from mice immunized in complete Freund's adjuvant (CFA) with S. aureus irradiated in the absence (IRS) or presence (MnDP-IRS) of Mn-DP-Pi. Data are representative of 2-3 independent experiments and show mean values from triplicate samples (panels a-c) or n=4 mice (panel d). Error bars reflect SEM (standard error of the mean).

FIG. 4 depicts prior MRSA skin infection not protecting against subsequent infection in the mouse model. Skin lesion size after MRSA infection of naïve mice or mice that had resolved a previous infection 2 weeks prior (n=4 mice/group). Data are representative of 3 independent experiments and shows mean+/−SEM.

FIG. 5 Immunization with a Mn-DP-Pi-based irradiated MRSA vaccine protects against staphylococcal skin infection. (a) Skin lesions in wild-type (WT) mice. 3 representative mice/group are shown with lesions demarcated in red. Lesions were photographed 6 days after challenge, which occurred 14 days after the last immunization. (b) Daily lesion area in WT mice. (c) Skin MRSA burden in WT mice. Colony forming units (CFU) were determined 6 days after challenge. (d,e) WT or B cell-deficient (pMT) mice were immunized and challenged as in panels a-c. Lesion area (d). Skin CFU (e). In all studies, mice were immunized with IRS or MnDP-IRS in PBS or CFA on day 0 and day 14. On day 28, mice were challenged subcutaneously with 10⁷ CFU of live MRSA. Control antibody (CTRL Ab) or depleting antibody against CD4 T cells (αCD4) was administered to the indicated groups 24 hours prior to challenge. Data are representative of 2-3 independent experiments. n=4-10 mice/group. Error bars reflect SEM. Asterisks indicate p value compared to unimmunized (panel b) or indicated (panels c and e) group (*p<0.05, **p<0.01).

FIG. 6 depicts that Alum is an effective adjuvant for the MnDP-IRS vaccine. (a-b) Lesion size and skin CFU in WT mice that were challenged after immunization with IRS or MnDP-IRS in alum as described in FIG. 4 for CFA (n=8-9 mice/group). Data are shown as mean+/−SEM. Abbreviations as in FIG. 4.

FIG. 7 depicts the structural integrity and adsorption of λ phage assessed by transmission electron microscopy. (a) Non-irradiated λ phage. I, purified phage; II, adsorption to E. coli. (b) λ phage exposed to 40 kGy in the absence or presence of Mn-DP-Pi. I, post-irradiation in liquid-holding (4° C.); II, frozen (−80° C.) after irradiation, then thawed. (c) Binding between λ phage tail and E. coli. I, adsorption of non-irradiated λ phage to E. coli (left, low magnification) and a single bound phage (right, high magnification); II, adsorption of λ phage exposed to 40 kGy in Mn-DP-Pi to E. coli (left) and a single bound phage (right). Note, the structural integrity of phage tails exposed to 40 kGy in Pi buffer was lost (panel b of this figure and FIG. 1c), so their ability to adsorb to E. coli was not tested. λ phage heads are 50 nm in diameter; λ tails are 150 nm long. Abbreviations as in FIG. 1.

### DETAILED DESCRIPTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, and materials are described.

As used herein, “a” or “an” means at least one, unless clearly indicated otherwise. The term “about,” unless otherwise indicated, refers to a value that is no more than 10% above or below the value being modified by the term. For example, the term “about 5% (w/w)” means a range of from 4.5% (w/w) to 5.5% (w/w).

The inventors have studied the radio-resistance of D. radiodurans and prepared ultra-purified, protein free-cell extracts that exhibit radioprotective properties. Thus, the invention is based in part on the discovery of radioprotective components of D. radiodurans cell free extract and artificial compositions containing such components.

In particular, applicants have shown that D. radiodurans ultra-purified and protein-free cell extracts are extremely radioprotective of proteins exposed to gamma-radiation. Adenosine, uridine and peptides are accumulated in D. radiodurans ultrafiltrate at higher concentrations than in ultrafiltrates of radiation sensitive bacteria. In vitro, at doses >10,000 Gy, nucleosides were shown to be highly protective of proteins, preventing ionizing radiation (IR)-induced protein carbonylation and preserve the function enzymes in the presence of Mn(II). A radioprotective composition of adenosine, manganese, peptides and phosphate has been developed. Surprisingly, D. radiodurans extracts have been shown to be potent radioprotectors for cultured human T-cells with greater potency than other well-established radioprotective compounds.

The present invention provides for radioprotective compositions either synthetic or derived from D. radiodurans (DR) and methods of uses of these compositions to protect at least the structure of proteins from radiation damage. The composition of the present invention comprise manganese and at least one antioxidant peptide, or they comprise manganese and a collection of individual amino acids. In additional embodiments, the composition may also comprise at least one nucleoside. As used herein, the term “radioprotective composition” or “radiation protective composition” can mean either a DR ultrafiltrate extract prepared according to methods described herein, or it can mean a synthetic composition comprising manganese and at least one antioxidant peptide or a collection of individual amino acids. If a DR ultrafiltrate extract is used, this extract can be supplemented with any of the compounds described and disclosed herein. For example, the DR ultrafiltrate may be prepared according to the methods disclosed herein, and additional Mn²⁺ or peptides, for example, may be added to the extract.

The radioprotective compositions may further contain leucine, alanine, and/or valine. Leucine is strongly implicated in scavenging hydrogen peroxide in the presence of Mn(II), and may be components of larger intracellular complexes that include uridine and adenosine. Strong in vitro evidence indicates a synergistic effect between adenosine and manganese and phosphate. The stoichiometry of adenosine and manganese and phosphate or bicarbonate buffers may be optimized for an apoptosis assay.

Although not being bound by any particular theory, it is believed that compositions comprising purine nucleosides (e.g. adenosine), pyrimidine nucleosides (e.g., uridine) and a peptide antioxidant (e.g. manganese-peptide) act as radioprotectants by shielding a proteins' active site and surface. The purine nucleoside e.g. adenosine (and optionally combined with the pyrimidine nucleoside uridine, and peptides) mediates its radioprotective effects upon accumulation within a cell, which inhibits radiation-induced protein oxidation, and in the presence of Mn(II) preserves enzyme function. Adenosine is thought to protect proteins, and therefore scavenge a subset of ROS.

This invention provides for methods of preserving protein function or protein immunogenicity comprising contacting a protein with a composition of the present invention. One embodiment of the invention is a method preserving protein function or protein immunogenicity when the protein is exposed to the extreme conditions of radiation such as e.g. gamma radiation. In another embodiment of the invention, the method preserves protein function or protein immunogenicity during desiccation. In one embodiment, the proteins that are protected are comprised within and/or on a cell.

The methods of preserving protein function or protein immunogenicity provide radioprotection when the protein is exposed to high dose of radiation such as doses in excess of 10 kGy, e.g., 17.5 kGy.

In another embodiment, the invention provides for methods of protecting protein function or protein immunogenicity in a cell or virus culture comprising culturing, harvesting and/or suspending the cells or viruses with any of the radio-protective compositions described herein. The cell culture may be bacterial. In one embodiment, the cell culture is methicillin-resistant Staphylococcus aureus (MRSA). The culture comprising a virus culture may be bacteriophage lambda or Venezuelan equine encephalitis virus (VEEV).

Any nucleoside, if present, may be used in the radiation protective compositions. Suitable nucleosides include, but are not limited to, adenosine, uridine, β-pseudouridine, inosine, and mixtures thereof. In addition, analogues of nucleosides containing two carbonyl oxygen groups (C═O) separated by one (N3)H group can also be used. In one embodiment, the nucleoside is adenosine or uridine. In one embodiment, the composition contains adenosine. In other embodiment of the invention, the composition contains uridine. The amount of nucleoside in the composition varies on its use. Those of skill in the art will be able to determine the suitable amount. In some embodiments of the invention, the amount of nucleoside ranges from about 0.01 mM to about 15 mM, from about 0.1 mM to about 1 mM, from about 1 mM to about 10 mM, from about 1 mM about 15 mM. In one embodiment, the concentration of one or more nucleosides comprises about 1 mM to about 15 mM of adenosine and/or uridine.

A variety of antioxidants may be used or present in the composition. Suitable antioxidants include manganese, vitamin E and manganous phosphate, Mn-peptides, Mn-amino acids (e.g., Leucine), Mn-TRIS, Mn-melanin, Mn-caffeine, Mn-ribose, Mn-trehalose, Mn-dipicolinic acid, Mn-phosphate and Mn-bacarobonate. In one embodiment of the invention, the antioxidant is manganese. In another embodiment, the antioxidant is MnCl₂. In yet another embodiment, the antioxidant is vitamin E and/or aspirin. The amount of antioxidant in the composition varies in its use. Those of skill in the art will be able to determine the suitable amount. In one embodiment, the composition contains about 0.01 mM to about 15 mM of the antioxidant. In another embodiment, the composition contains about 0.01 mM to about 12.5 mM.

In one embodiment of the invention, one antioxidant is manganous phosphate which may be provided as a mixture. In one embodiment the mixture is produced by mixing a solution of manganese and a solution of phosphate. The amount of antioxidant in the composition varies on its use. Those of skill in the art will be able to determine the suitable amount. In one embodiment, the compositions comprise from about 0.01 mM to about 15 mM of the manganous (Mn(II)) ions. In a more specific embodiment, the compositions comprise from about 0.01 mM to about 15 mM of the manganous (Mn(II)) ions in a phosphate buffer. In a still more specific embodiment, the compositions comprise phosphate buffer at a concentration of from about 1 mM to about 25 mM. In one specific embodiment, the mixture is a 1 mM solution of Mn(II) and a solution of 25 mM phosphate buffer (ph 7.4).

The compositions contain one or more amino acids that exhibit cytoprotective properties. In one embodiment of the invention, composition further contains at least one or more amino acid selected from the group consisting of asparagine, glutamine, serine, histidine, glycine, threonine, arginine, tyrosine, methionine, phenylalanine, isoleucine, lysine, ornithine, leucine, valine and alanine. In another embodiment, the amino acid is leucine. In an alternate embodiment, the amino acid is glycine. In another embodiment, the compositions include at least leucine and alanine. In another embodiment, the composition does not contain proline. In still another embodiment, the composition contains 10% or less proline as measured against the presence of another amino acids. For example, an equal mixture of 12 distinct amino acids would contain 1 proline residue or less in this embodiment.

As an alternative, or in addition to the presence of individual amino acids, the compositions and the methods using these compositions may comprise at least one small peptide such as, but not limited to, a decpeptide. As used herein, “small peptide” means a small, linear chain of amino acids of no more than about 25 residues in length. In one embodiment, the small peptides used in the compositions or methods of the present invention are about 25, 24, 23, 22, 21, 20, 19, 18, 17, 16, 15, 14, 13, 12, 11, 10, 9, 8, 7, 6, 5, 4, 3 or 2 amino acids in length. In one embodiment, the compositions and methods using these compositions may comprise at least one small peptide, wherein the small peptide comprises an amino acid sequence that is at least about 80% identical to the amino acid sequence of SEQ ID NO:1: Asp-Glu-His-Gly-Thr-Ala-Val-Met-Leu-Lys (SEQ ID NO:1), a decapeptide herein referred to as “DP” or “the decapeptide.” In one embodiment, the small peptide contains no proline residues. In another embodiment, the peptide contains less that 10% of proline residues as compared to other amino acids. For example, in this specific embodiment, a 12-mer would contain one proline residue or less.

In still further embodiments, each of the small peptides independently comprise an amino acid sequence at least 60%, 70%, 80%, 85%, 86%, 87%, 88%, 89%, 90%, 91%, 92%, 93%, 94%, 95%, 96%, 97%, 98%, 99% or 100% identical to the amino acid sequence of SEQ ID NO:1. Small peptides that are less than 100% identical to the amino acid sequence of SEQ ID NO:1 are considered variants thereof.

The amount of small peptide will vary. Those of skill in the art will be able to determine the suitable amount depending on a variety of factor such as the subject, the duration of the radiation exposure, the amount of the radiation exposure, etc. In some embodiments of the invention, the amount of small peptide ranges from about 0.01 mM to about 15 mM, from about 0.1 mM to about 1 mM, from about 1 mM to about 10 mM, from about 1 mM about 15 mM. In one embodiment, the concentration of one or more small peptide comprises about 1 mM to about 15 mM of the peptide of SEQ ID NO:1 or variants thereof. In other embodiments, the concentration of one or more small peptides comprises about 15 mM or less, about 14 mM or less, about 13 mM or less, about 12 mM or less, about 11 mM or less, about 10 mM or less, about 9 mM or less, about 8 mM or less, about 7 mM or less, about 6 mM or less, about 5 mM or less, about 4 mM or less, about 3 mM or less, about 2 mM or less, about 1 mM or less or about 0.5 mM or less of the peptide of SEQ ID NO:1. Of course, the concentration of one or more small peptides can be in between any of the listed concentrations, for example between about 15 mM and about 14 mM, between about 14 mM and about 13 mM, between about 13 mM and about 12 mM, between about 12 mM and about 11 mM, between about 11 mM and about 10 mM, between about 10 mM and about 9 mM, between about 9 mM and about 8 mM, between about 8 mM and about 7 mM, between about 7 mM and about 6 mM, between about 6 mM and about 5 mM, between about 5 mM and about 4 mM, between about 5 mM and about 3 mM, between about 3 mM and about 2 mM, between about 2 mM and about 1 mM, between about 1 mM and about 0.5 mM, etc of the peptide of SEQ ID NO:1 or variants thereof.

A polypeptide having an amino acid sequence at least, for example, about 95% “identical” to a reference amino acid sequence, e.g., SEQ ID NO:1, is understood to mean that the amino acid sequence of the polypeptide is identical to the reference sequence except that the amino acid sequence may include up to about five modifications per each 100 amino acids of the reference amino acid sequence. In other words, to obtain a peptide having an amino acid sequence at least about 90% identical to a reference amino acid sequence, up to about 10% of the amino acid residues of the reference sequence may be deleted or substituted with another amino acid or a number of amino acids up to about 10% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

As used herein, “identity” is a measure of the identity of nucleotide sequences or amino acid sequences compared to a reference nucleotide or amino acid sequence. In general, the sequences are aligned so that the highest order match is obtained. “Identity” per se has an art-recognized meaning and can be calculated using published techniques. (See, e.g., Computational Molecular Biology, Lesk, A. M., ed., Oxford University Press, New York (1988); Biocomputing: Informatics And Genome Projects, Smith, D. W., ed., Academic Press, New York (1993); Computer Analysis of Sequence Data, Part I, Griffin, A. M., and Griffin, H. G., eds., Humana Press, N.J. (1994); von Heinje, G., Sequence Analysis In Molecular Biology, Academic Press (1987); and Sequence Analysis Primer, Gribskov, M. and Devereux, J., eds., M Stockton Press, New York (1991)). While there are several methods to measure identity between two polynucleotide or polypeptide sequences, the term “identity” is well known to skilled artisans (Carillo, H. & Lipton, D., Siam J Applied Math 48:1073 (1988)). Methods commonly employed to determine identity or similarity between two sequences include, but are not limited to, those disclosed in Guide to Huge Computers, Martin J. Bishop, ed., Academic Press, San Diego (1994) and Carillo, H. & Lipton, D., Siam J Applied Math 48:1073 (1988). Computer programs may also contain methods and algorithms that calculate identity and similarity. Examples of computer program methods to determine identity and similarity between two sequences include, but are not limited to, GCG program package (Devereux, J., et al., Nucleic Acids Research 12(i):387 (1984)), BLASTP, ExPASy, BLASTN, FASTA (Atschul, S. F., et al., J Molec Biol 215:403 (1990)) and FASTDB. Examples of methods to determine identity and similarity are discussed in Michaels, G. and Garian, R., Current Protocols in Protein Science, Vol 1, John Wiley & Sons, Inc. (2000), which is incorporated by reference.

In one embodiment of the present invention, the algorithm used to determine identity between two or more polypeptides is BLASTP. In another embodiment of the present invention, the algorithm used to determine identity between two or more polypeptides is FASTDB, which is based upon the algorithm of Brutlag et al. (Comp. App. Biosci. 6:237-245 (1990), incorporated by reference). In a FASTDB sequence alignment, the query and reference sequences are amino sequences. The result of sequence alignment is in percent identity. Parameters that may be used in a FASTDB alignment of amino acid sequences to calculate percent identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent identity. For query sequences truncated at the N- or C-termini, relative to the reference sequence, the percent identity is corrected by calculating the number of residues of the query sequence that are N- and C-terminus to the reference sequence that are not matched/aligned, as a percent of the total bases of the query sequence. The results of the FASTDB sequence alignment determine matching/alignment. The alignment percentage is then subtracted from the percent identity, calculated by the above FASTDB program using the specified parameters, to arrive at a final percent identity score. This corrected score may be used for the purposes of determining how alignments “correspond” to each other, as well as percentage identity. Residues of the reference sequence that extend past the N- or C-termini of the query sequence may be considered for the purposes of manually adjusting the percent identity score. That is, residues that are not matched/aligned with the N- or C-termini of the comparison sequence may be counted when manually adjusting the percent identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent identity score calculated by the FASTDB program. If the remaining 90 residues were perfectly matched (100% alignment) the final percent identity would be 90% (100% alignment−10% unmatched overhang). In another example, a 90 residue query sequence is compared with a 100 reference sequence, except that the deletions are internal deletions. In this case the percent identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent identity. The addition in the query occurs at the N-terminus of the query sequence and therefore, the FASTDB alignment may not show a match/alignment of the first 10 residues at the N-terminus. If the remaining 100 amino acid residues of the query sequence have 95% identity to the entire length of the reference sequence, the N-terminal addition of the query would be ignored and the percent identity of the query to the reference sequence would be 95%.

In one embodiment, the compositions comprise adenosine, uridine, leucine, adenine, and manganese. In another embodiment, the composition comprises about 1 to about 15 mM adenosine and about 1 to about 12.5 mM MnCl₂. In another embodiment, the composition comprises a D. radiodurans extract containing one or more nucleosides and one or more antioxidants.

Bacterial vaccines comprise a minority of licensed vaccines (Plotkin S, Vaccines, 5^{th} ed.). This likely reflects some unique challenges posed by bacterial, as compared to viral, targets for vaccine development. Although killed bacterial vaccines against typhoid, cholera, and plague appeared in the 19^{th} century shortly after Jenner's smallpox and Pasteur's rabies introduced the world to viral vaccines, these were later replaced in the 20^{th} century with live attenuated vaccines against typhoid and cholera to capitalize on the enhanced immunogenic properties of live bacteria. However, the potential reversion to virulence of live attenuated organisms prevents this strategy from being enthusiastically embraced.

The more recent bacterial vaccines developed in the latter 20^{th} and early 21^{st} century mainly consist of capsular polysaccharides conjugated to carrier proteins. This technique of using capsular polysaccharides, however, has proven effective against bacteria only for which the polysaccharide capsule is a key virulence factor and whose neutralization using antibodies is effective in preventing infection (e.g. Streptococcus pneumoniae, Haemophilus influenzae type b, Neisseria meningitides). The capsular polysaccharide strategy has failed against most other bacteria, most notably Staphylococcus aureus, which utilize other virulence strategies.

This failure to develop a neutralizing antibody against bacteria that utilize other virulence strategies highlights a major challenge posed by bacteria to vaccine development: their larger genome compared to viruses allows them to produce multiple and more complex structural and virulence factors such that neutralizing a single factor with a vaccine is often not sufficient for protective immunity. Furthermore, the multiple and complex virulence strategies of bacteria may require multiple immune mechanisms, i.e. both cellular and humoral immunity, to achieve protection. Irradiated whole bacteria, i.e., killed bacteria, present a safe yet immunogenic strategy to achieve multiple mechanisms of immune protection against multiple bacterial epitopes.

The invention therefore provides methods of producing vaccines directed against microorganisms, with the methods comprising culturing, harvesting, and/or suspending the microorganism in the presence of a radiation-protective composition of the present invention and irradiating the bacteria with a dose of radiation sufficient to render the microorganism replication-deficient. In one embodiment, the radiation protective composition is synthetic; in another embodiment, the radiation protective composition is DR ultrafiltrate extract. In one embodiment, the vaccine is directed against methicillin-resistant Staphylococcus aureus (MRSA). In another embodiment, the vaccine is directed against Venezuelan equine encephalitis virus (VEEV).

Methods of vaccine preparation are well known in the art. The methods provided herein can be applied to these well-known vaccine preparation methods, or they can be used separately and apart from traditional vaccine preparation methods. For example, one embodiment of the present invention provides for methods of vaccine preparation without genetically engineering the microorganism against which the vaccine is being prepared. The methods disclosed herein allow for normal, wild-type microorganisms to be cultured, harvested, and/or suspended in the presence of the radiation-protective compositions, such that the three-dimensional structure of the proteins within and the cell surface markers on the microorganisms is preserved during an extreme dose of radiation. The dose of radiation is designed to obliterate the genome of the microorganism such that the microorganism is incapable of replication. After dosing with radiation, the replication-deficient cells can be collected and vaccine preparation can be carried out using normal vaccine preparatory techniques. The protective compositions of the present invention preserve at least a fraction of the immunogenic proteins of the microorganism, such that administration of a vaccine comprising the irradiated microorganism to an animal will produce an immunogenic response. Thus, the present methods of vaccine preparation can be practiced using routine cell culture techniques. The microoganisms against which a vaccine can be prepared using the methods of the present invention include bacteria and viruses. Standard cell culture techniques for bacteria and viruses are well known in the art.

Of course, the vaccine preparation methods of the present invention are not limited to a particular type of radiation, provided the type and dose used is capable of rendering the microorganism replication defective. Examples of radiation include but are not limited to, UV light, alpha radiation, beta radiation, gamma radiation, X-ray radiation and neutron radiation. In one embodiment, the dose of radiation is at least about 20 kGy. The dose of radiation may be over 25,000 Gy (25 kGy) for bacterial mixtures and the dose of radiation may be over 40,000 Gy (40 kGy) for viral mixtures.

Accordingly, the present invention provides a vaccine comprising irradiated methicillin-resistant Staphylococcus aureus (MRSA), wherein the irradiated MRSA is antigenic when administered to a subject capable of generating an immune response. The vaccine is prepared according to the methods disclosed herein such that the MRSA is replication deficient. The membrane proteins of the irradiated MRSA, however, enough intact to elicit an immune response from the subject.

Accordingly, the present invention provides a vaccine comprising irradiated Venezuelan equine encephalitis virus (VEEV), wherein the irradiated VEEV is antigenic when administered to a subject capable of generating an immune response. The vaccine is prepared according to the methods disclosed herein such that the VEEV is replication deficient. The envelope proteins of the irradiated VEEV, however, enough intact to elicit an immune response from the subject.

The invention also provides methods of treating a subject in need of treatment of a bacterial or viral infection. In one specific embodiment, the bacterial infection is methicillin-resistant Staphylococcus aureus (MRSA). In another specific embodiment, the viral infection is Venezuelan equine encephalitis virus (VEEV). The invention also provides methods of reducing the likelihood of acquiring a bacterial or viral infection. In one specific embodiment, the bacterial infection is methicillin-resistant Staphylococcus aureus (MRSA). In another specific embodiment, the viral infection is Venezuelan equine encephalitis virus (VEEV)

A “subject in need of treatment” is an animal with a bacterial or viral infection that may or may not exhibit symptoms of the infection. The animal can be a fish, bird, or mammal. Exemplary mammals include humans, domesticated animals (e.g., cows, horses, sheep, pigs, dogs, and cats), and exhibition animals, e.g., in a zoo. In a preferred embodiment, the subject is human.

The terms “treating”, “treatment,” and “therapy” as used herein refer to curative therapy, prophylactic therapy, and preventative therapy.

As used herein, unless stated otherwise, the terms vaccine or vaccine composition are meant to encompass, and not limited to, pharmaceutical compositions and nutraceutical compositions containing the irradiated microorganisms of the invention. The vaccines may also contain one or more “excipients” that are “inactive ingredients” or “compounds” devoid of pharmacological activity or other direct effect in the diagnosis, cure, mitigation, treatment, or prevention of disease or to affect the structure or any function of the human body.

The vaccines may also comprise adjuvants. Suitable adjuvants will be well known to those of skill in the art. Exemplary adjuvants include complete or incomplete Freund's adjuvant, RIBI (muramyl dipeptides), ISCOM (immunostimulating complexes) and aluminum hydroxide (Alum).

The methods of the instant application are particularly advantageous. Compared to well-established radioprotectors (such e.g. amifostine), compositions comprising one or more nucleosides and one or more antioxidants (e.g., adenosine, uridine, peptides and Mn) are relatively non-toxic, thus the vaccine preparations may not need to be purified to any extent prior to administration.

The invention also provides methods of rendering proteins bacteria or viruses in culture resistant to ionizing radiation (IR), with these methods comprising culturing the bacteria or virus in the presence of a radiation-protective compositions of the present invention. The radiation-protective compositions used in IR-resistant methods of the present invention comprise at least the DP peptide as described herein (or a variant thereof), phosphate, at least one antioxidant and any non-metabolizable hydroxyl-radical scavengers, such as but not limited to, dimethyl sulfoxide (DMSO).

While these vaccination methods may be used in any mammalian species such as farm animals including, but not limited to, horses, sheep, pigs, chicken, and cows, the preferred use of compositions is for a human.

The effective dosage rates or amounts of the vaccination compositions will depend in part on whether the vaccination will be used therapeutically or prophylactically, the duration of exposure of the recipient to radiation, the type of radiation, the size, and weight of the individual, etc. The duration for use of the vaccination also depends on whether the use is for prophylactic purposes. Any dosage form employed should provide for a minimum number of units for a minimum amount of time.

Selection of the preferred effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration and should be decided according to the judgment of the practitioner and each patient's circumstances. Effective doses may be extrapolated from dose-response curves derived from in vitro or animal model test systems.

The compositions of the present invention can be administered via parenteral, subcutaneous, intravenous, intramuscular, intraperitoneal, transdermal, or buccal routes. For example, an agent may be administered locally via microinfusion. The dosage administered will be dependent upon the age, health, and weight of the recipient, kind of concurrent treatment, if any, frequency of treatment, and the nature of the effect desired.

In one embodiment of the invention, the method comprises administration of the vaccine in a pharmaceutically acceptable carrier. Suitable carriers and their formulations are described in Remington's Pharmaceutical Sciences, 2005, Mack Publishing Co. Typically, an appropriate amount of a pharmaceutically acceptable salt is used in the formulation to render the formulation isotonic. Examples of the pharmaceutically acceptable carrier include liquids such as saline, Ringer's solution, and dextrose solution. The pH of the solution is preferably from about 5 to about 8, and more preferably from about 7 to about 7.5. It will be apparent to those persons skilled in the art that certain carriers may be more preferable depending upon, for instance, the route of administration and concentration of proinflammatory cytokine inhibitor being administered.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### EXAMPLES

### Example 1

### Preparation of Protein-Free Extract from D. radiodurans

D. radiodurans (ATTC BAA-816) was grown to OD600 0.9 in TGY, harvested by centrifugation, and lysed by French pressure treatment. The cells were washed and then lysed in double-distilled, de-ionized sterile water (dH₂O). Prior to lysis, cell density was adjusted with dH₂O to yield lysates representing approximately 50% intracellular concentration. Crude cell extracts were centrifuged for 20 hours at 175,000×g. The supernatant was passed through a <3 kiloDalton Microcon centrifugal filter (Millipore, USA) and boiled for 30 min. The Coomassie (Bradford) protein assay was used to confirm the virtual absence of proteins in the ultra-purified extracts, which were aliquoted and stored at −80° C.

### Example 2

### Analysis of Protein-Free Extract from D. radiodurans

The ultrafiltered cell extracts were prepared from D. radiodurans (ATCC BAA-816), P. putida (ATCC 47054), E. coli (MG1655), and T. thermophilus (ATCC BAA-163). M. E. Maguire provided wild-type E. coli (MM1925, strain K12) and its isogenic mntHmutant (MM2115). D. radiodurans recA- (rec30) and E. coli recA- (DH10B) are known in the art. The Jurkat T cell line was ATCC TIB-152. The DR-, PP-, EC- and TT-ultrafiltrates were prepared from bacteria grown as batch cultures in TGY medium to the same optical density at 600 nm (0.9; log-phase). For large-scale production of DR-ultrafiltrate used in the E. coli and Jurkat T cell radioprotection studies, high cell-density growth of D. radiodurans was in a 20 L fermentor. The cells were broken open by passage through a French Press. In the following order, bacterial lysates were centrifuged at 12,000×g (1 h, 4° C.); the supernatants were standardised for concentration on a protein-basis and ultracentrifuged at 190,000×g (48 h, 4° C.); and the ultracentrifuged supernatants were subjected to filtration through 3 kDa filters. The ultrafiltrates were boiled for 40 min, concentrated 5 times, and stored at −80° C. The chemical composition of the DR-, PP-, EC- and TT-ultrafiltrates were determined as follows: Mn and Fe on a Perkin Elmer model 4100ZL atomic absorption spectrometer; inorganic phosphate by the malachite green assay; bases, nucleosides and nucleotides by HPLC; protease activity with azocasein as substrate; and amino acids by pre-column derivatisation as implemented by Agent Technologies.

### Example 3

### The Reconstituted Mn²⁺ Peptide Complex

The extremely radioprotective Mn²⁺-decapeptide-phosphate complex is based on a consensus amino acid sequence (H-Asp-Glu-His-Gly-Thr-Ala-Val-Met-Leu-Lys-OH) (SEQ ID NO: 1) (“DP”) of hundreds of peptides purified from D. radiodurans. The composition of the mixture which spontaneously forms the Mn²⁺ complex comprises 3 mM (H-Asp-Glu-His-Gly-Thr-Ala-Val-Met-Leu-Lys-OH) (SEQ ID NO: 1), 1 mM MnCl₂, 25 mM orthophosphate (Pi) buffer (pH 7.4). When reconstituted in vitro, the Mn²⁺ complexes preserved the activity of enzymes exposed to 50,000 Gy. Studies with the decapeptides have demonstrated that it is the amino acid composition of the decapeptide, not the specific sequence of amino acids, which is critical to its radioprotective properties when combined with Mn²⁺ and orthophosphate buffer. The peptides need not be limited to 10 amino acids, but instead be comprised of the specific amino acids present in the above decapeptide.

### Example 4

### Application of reconstituted D. radiodurans Mn²⁺ complexes for the production of Irradiated Vaccines Against Bacteriophage Lamba

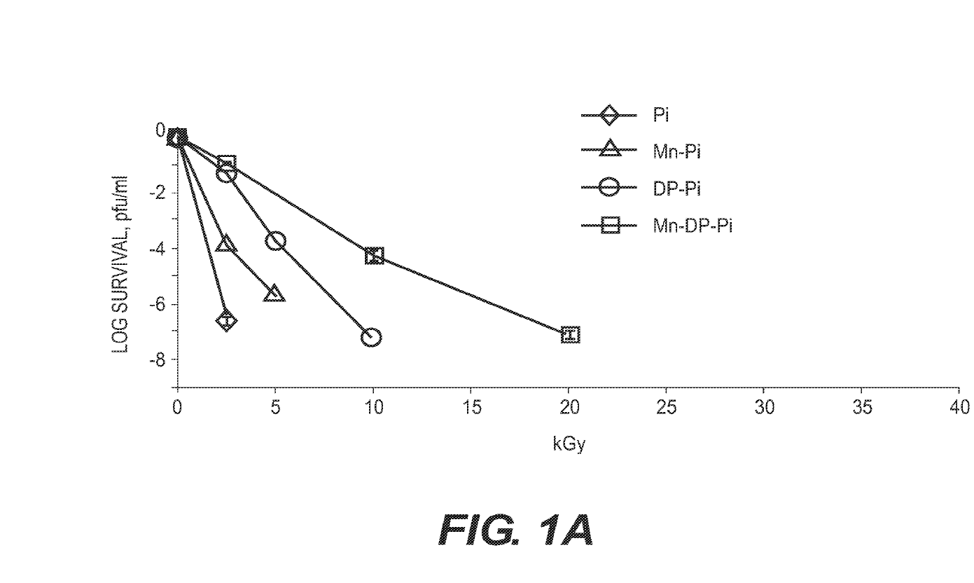

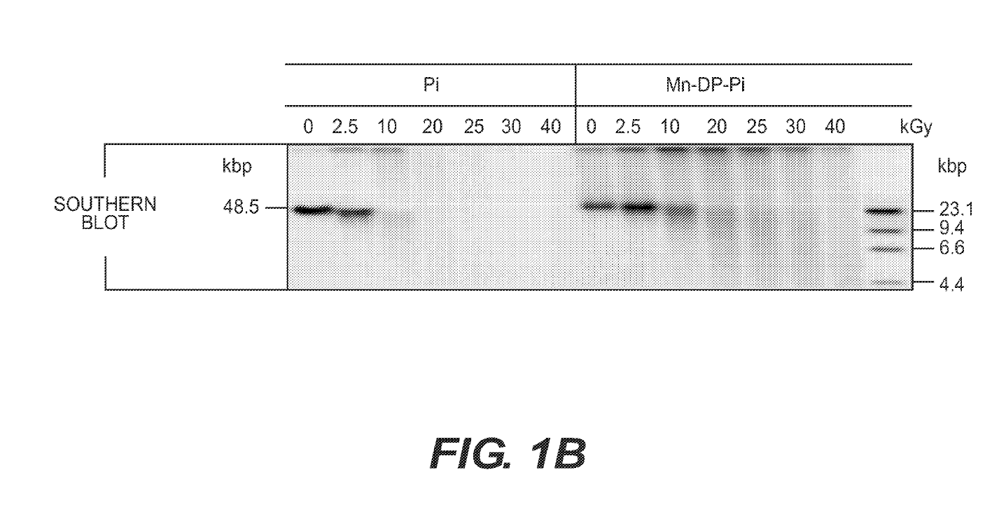

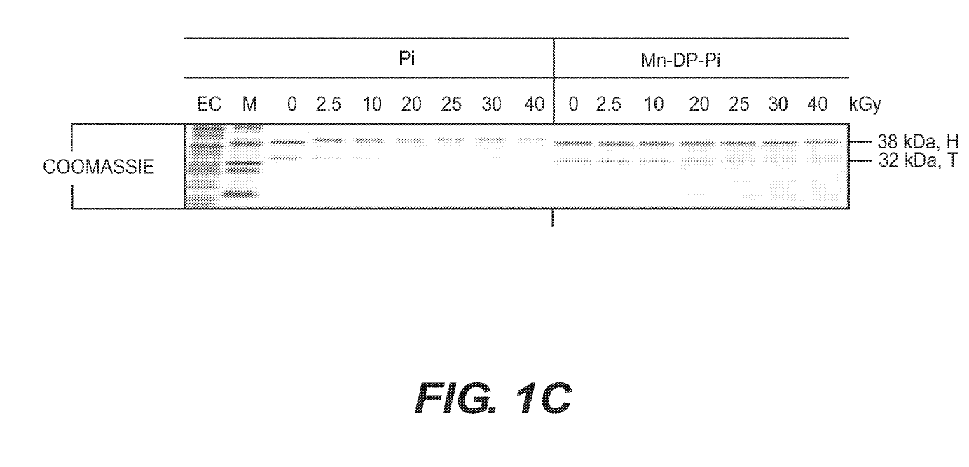

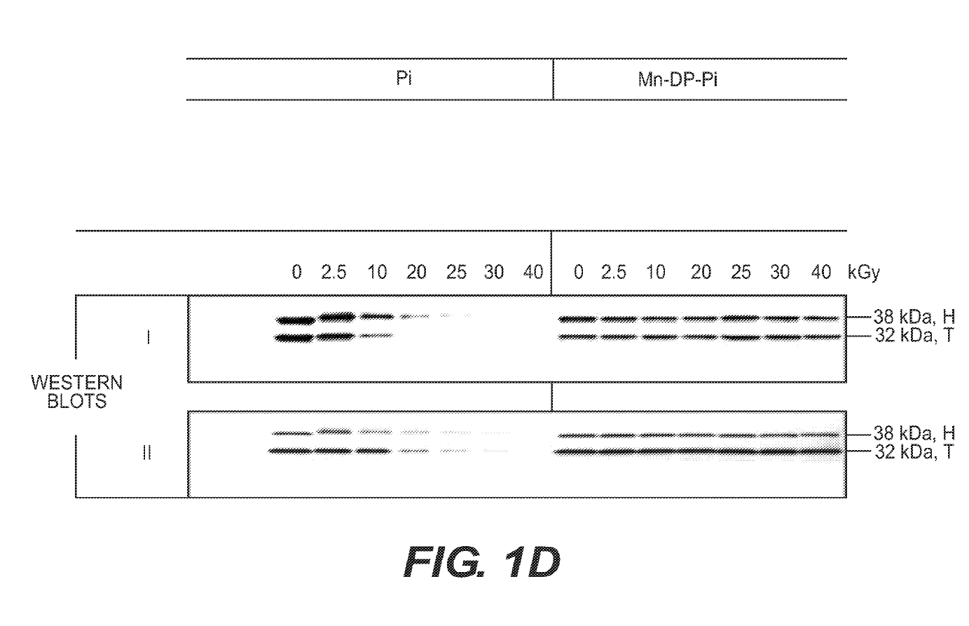

Irradiating bacteria using the methods described herein was tested and validated at 40,000Gy using the model bacteriophage Lambda virus (FIG. 1). DNA was prepared from irradiated bacteriophage λ treated or not with the Mn²⁺ complex (Mn-DP-Pi): 3 mM (H-Asp-Glu-His-Gly-Thr-Ala-Val-Met-Leu-Lys-OH) (SEQ ID NO: 1), 1 mM MnCl₂, 25 mM orthophosphate (Pi) buffer (pH 7.4). At the indicated gamma-ray doses (0-40 kGy), DNA (48.5 kbp genome) was purified from bacteriophage λ, subjected to conventional agarose gel electrophoresis, and then to Southern blotting with a radiolabelled 2 DNA probe. As shown in FIG. 1A, the Mn²⁺ complex does not significantly protect DNA packaged in viruses.

The same bacteriophage 2 preparations as examined in FIG. 1A were tested for protein integrity by separating the virus proteins using polyacrylamide gel electrophoresis. As shown in FIG. 1B, proteins in viruses which were irradiated in the absence of the Mn²⁺ complex were progressively destroyed. In contrast, the proteins in the virus samples which contained the Mn²⁺ complex were not affected by doses as high as 40 kGy.

At 40,000 Gy, a dose which obliterated the virus DNA (see FIG. 1A) and rendered the virus completely non-infective, the virus proteins remained fully immunogenic. This was tested by Western analysis, whereby λ proteins were challenged with antibodies raised in rabbits against non-irradiated λ phage. An identical positive result for immunogenicity was obtained for equivalent Westerns probed with antibodies raised against λ phage exposed to 40,000 Gy in the presence of the Mn²⁺ complex. In contrast, λ phage exposed to 40,000 Gy in the absence of the Mn²⁺ complex did not yield antibodies in rabbits which had significant specificity for native bacteriophage λ.

### Example 5

### Application of Reconstituted D. radiodurans Mn²⁺ Complexes for the Production of Irradiated Vaccines Against Methicillin Resistant Staphylococcus aureus

The approach in Example 5 above was also successfully tested on a pathogenic methicillin-resistant Staphylococcus aureus strain (MRSA). In contrast, viruses and bacteria exposed to supralethal doses of IR without the Mn²⁺ complexes resulted in substantial loss of viral epitope integrity and loss in immunogenicity.

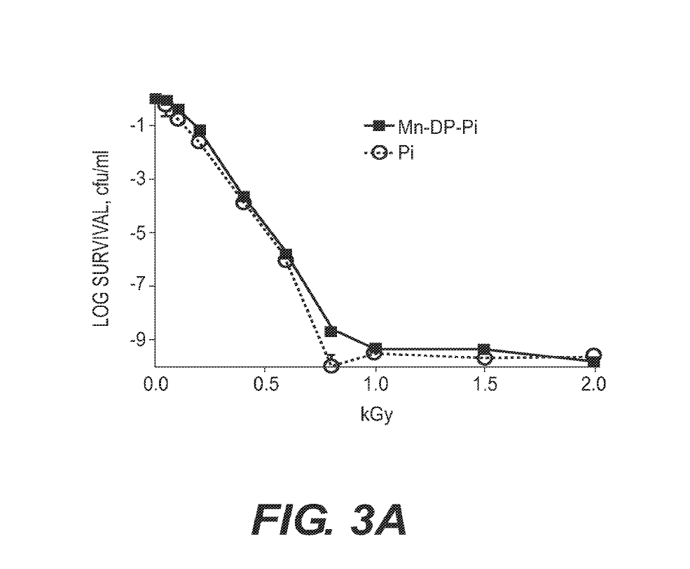

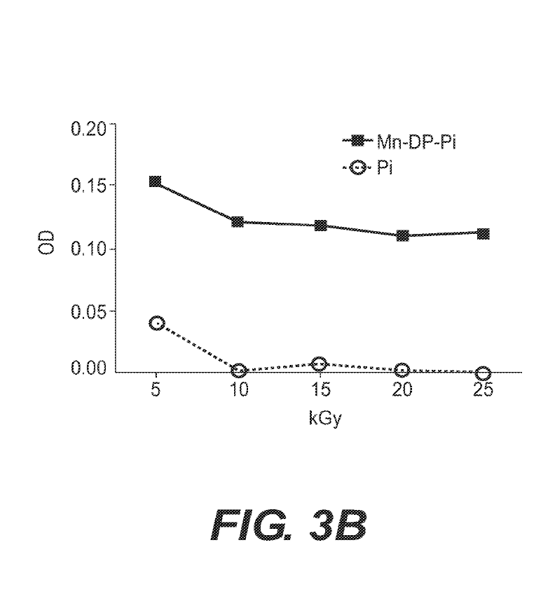

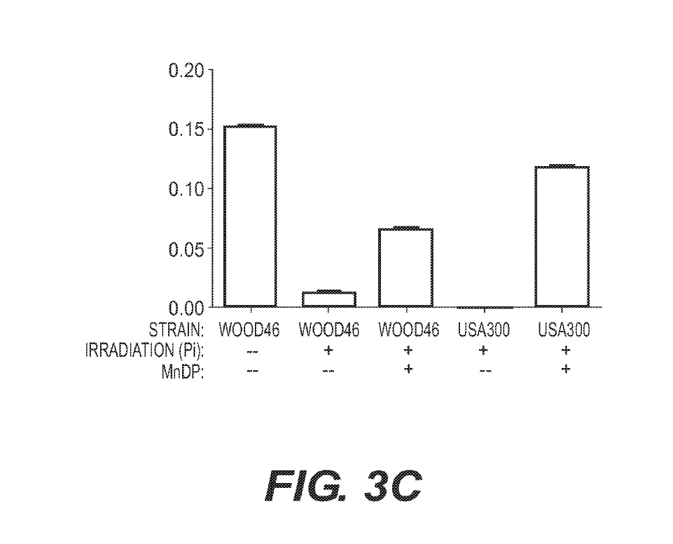

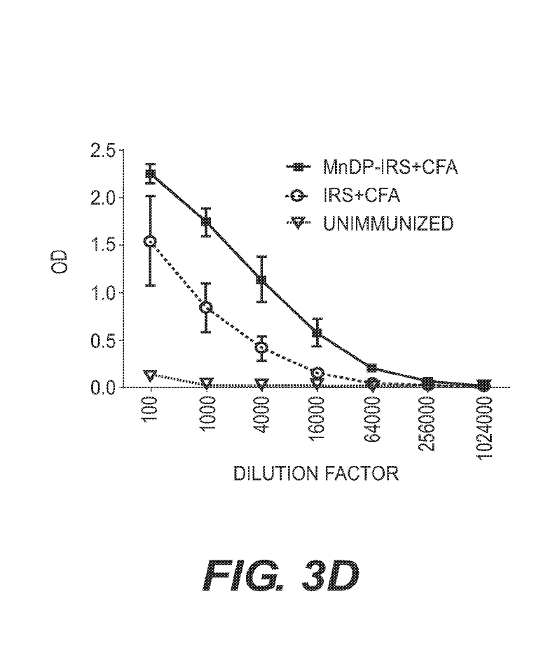

To evaluate the effects of Mn-DP-Pi on MRSA (strain USA300), the effect of Mn-DP-Pi on MRSA survival was tested. The radiation resistance of MRSA in Mn-DP-Pi was only slightly increased over irradiation in Pi buffer, with all viable cells killed by 2 kGy under both conditions (FIG. 3a). To assess if Mn-DP-Pi protected surface antigenic structures, plates were coated with MRSA that had been irradiated in the absence or presence of Mn-DP-Pi. The MRSA preparations were incubated with immune serum from mice previously infected with MRSA. Consistent with preservation of surface structures, MRSA irradiated in the presence of Mn-DP-Pi showed enhanced binding of anti-MRSA IgG (FIG. 3b). This was true at all levels of radiation exposure tested (up to 25 kGy), with only a 26% loss in binding signal between 5 and 25 kGy. In contrast, MRSA exposed to doses greater than 10 kGy in Pi buffer alone did not bind IgG above background levels (FIG. 3b).

MRSA USA300 expresses protein A, a virulence factor that binds the Fc portion of IgG molecules. The above results (FIG. 3b) do not distinguish between preservation of protein A and preservation of other antibody-binding epitopes on irradiated MRSA. Commercially available S. aureus bioparticles of inactivated Wood 46 strain, a strain that lacks protein A, was used to test for the preservation of other epitopes. Wood 46 exposed to 25 kGy in Mn-DP-Pi (MnDP-Wood 46) showed enhanced anti-MRSA IgG binding compared to Wood 46 irradiated in Pi buffer, confirming preservation of other antibody targets (FIG. 3c). Irradiated preparations of MnDP-Wood 46 and MnDP-USA300 bound less anti-MRSA IgG than non-irradiated preparations (FIG. 3c and data not shown). Irradiated MnDP-USA300 showed greater IgG binding than irradiated MnDP-Wood 46 (FIG. 3c), consistent with both Fc- and non-Fc-mediated binding of antibody by MnDP-USA300.

Next, mice were immunized with MRSA USA300 that had been exposed to 25 kGy in the absence (IRS, Irradiated Staphylococcus) or presence (MnDP-IRS) of Mn-DP-Pi. To test if epitope protection by Mn-DP-Pi translated to enhanced immune responses in vivo. Complete Freund's adjuvant (CFA) was included in immunizations. Compared to mice immunized with IRS, mice immunized with MnDP-IRS generated higher anti-S. aureus serum IgG titers (FIG. 3d). The higher levels of antibody in serum from mice immunized with MnDP-IRS (FIG. 3d) compared to serum from mice infected with live MRSA (FIG. 3b,c) further supported the immunogenicity of MnDP-IRS.

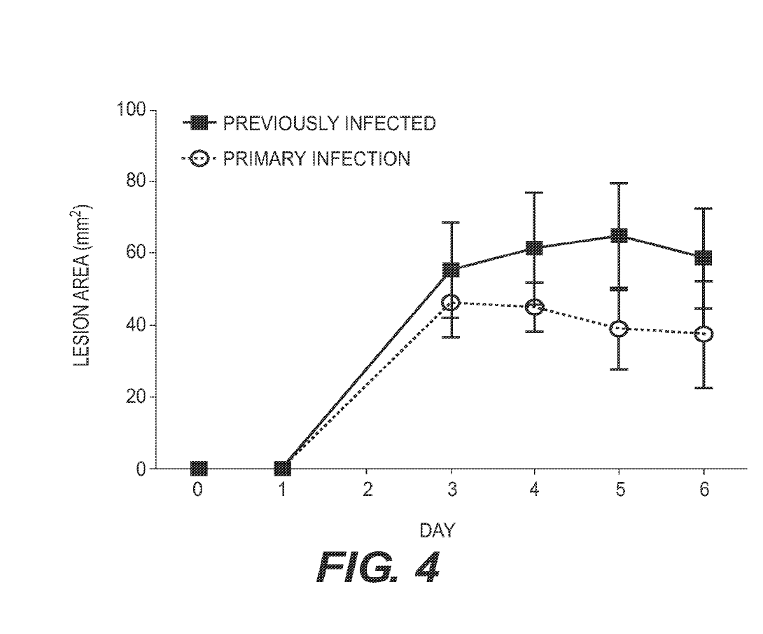

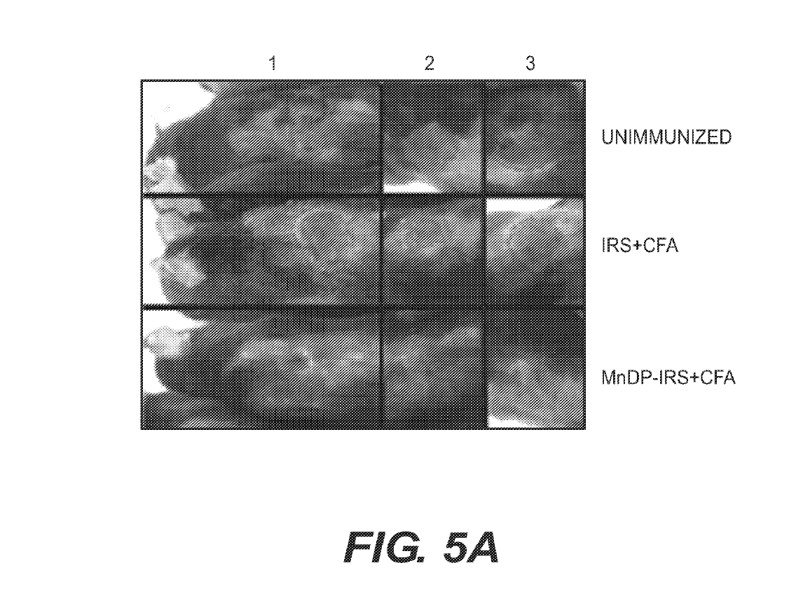

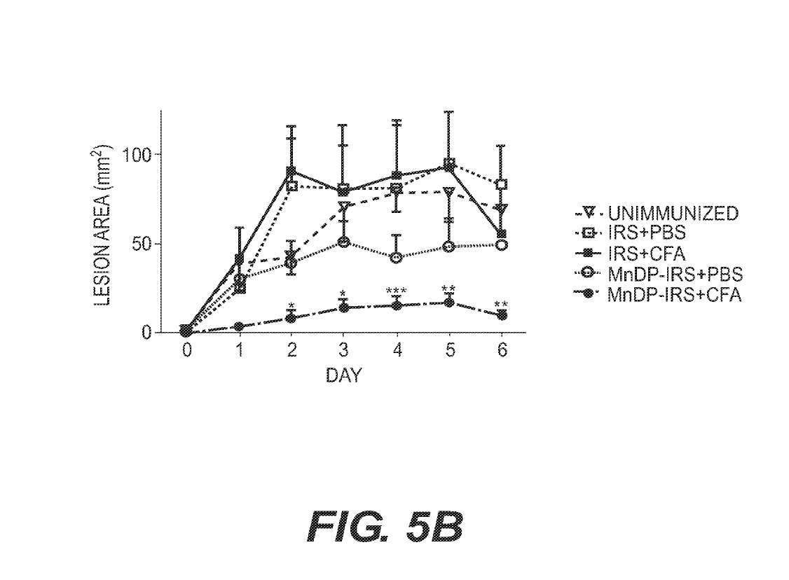

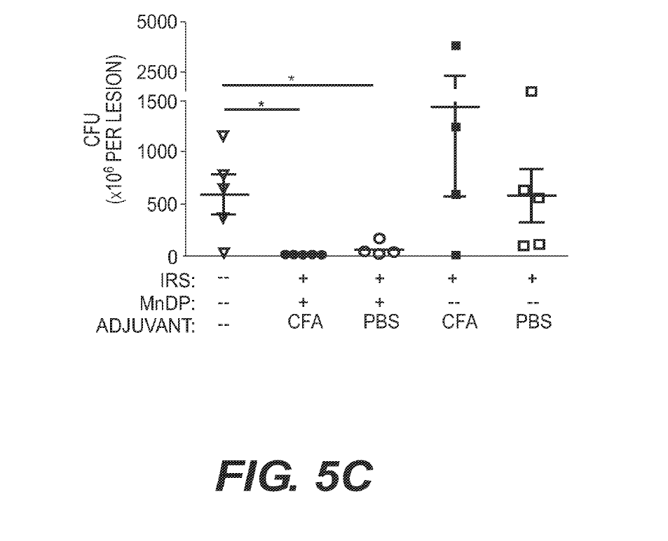

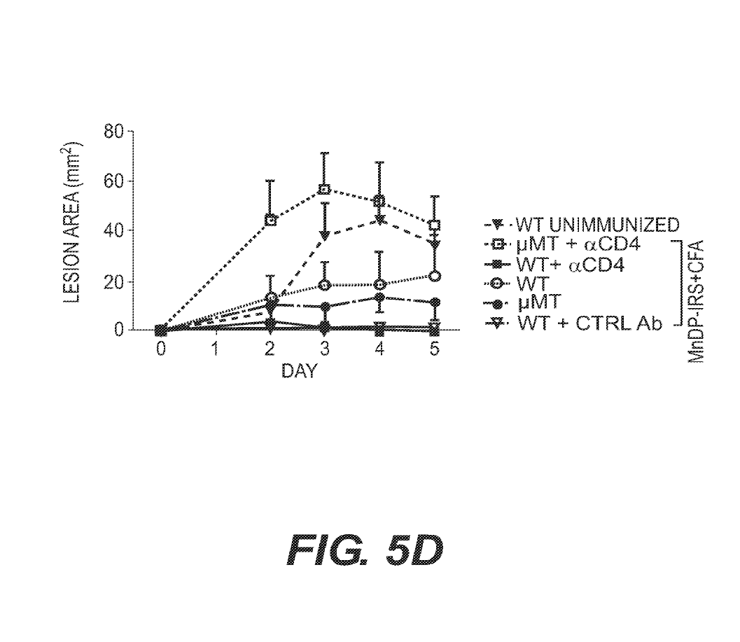

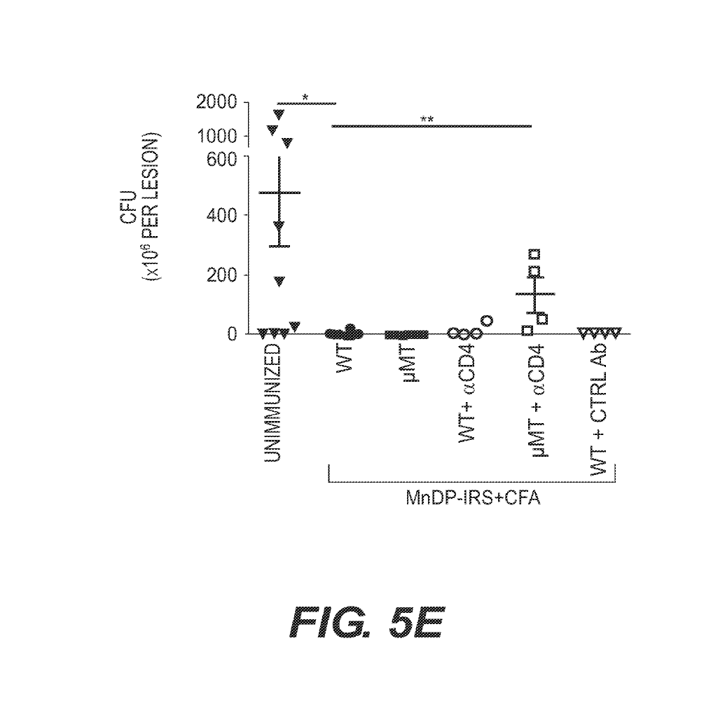

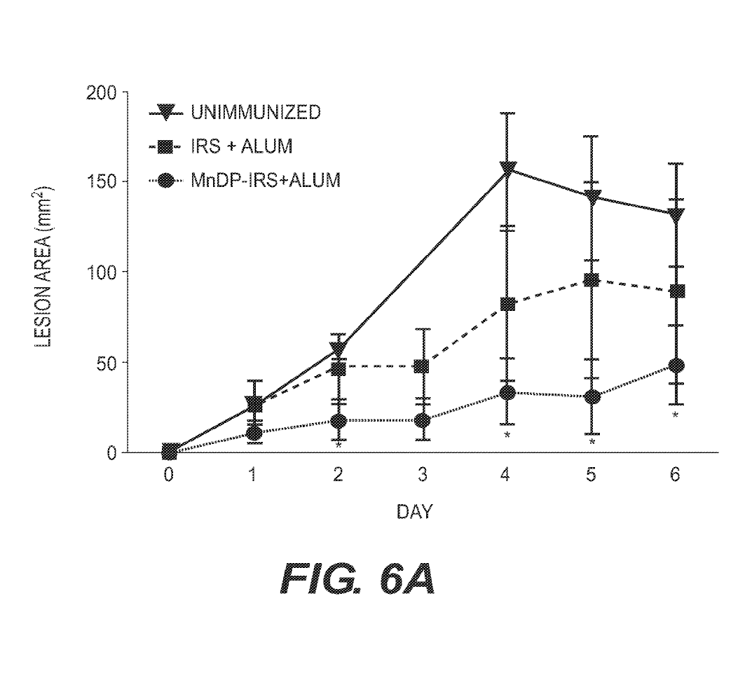

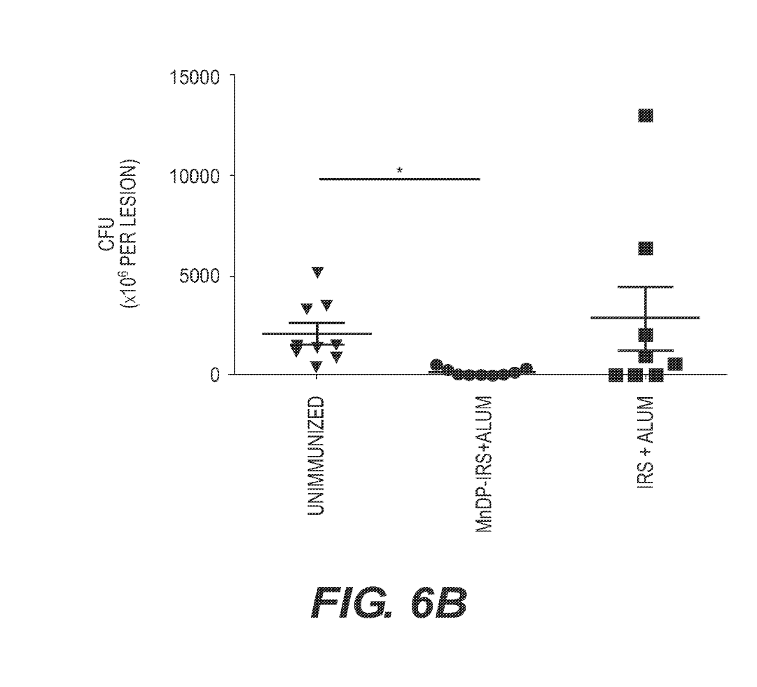

A mouse model of subcutaneous MRSA infection was used to evaluate the ability of MnDP-IRS immunization to elicit protective immunity. In this model, mice infected subcutaneously with MRSA USA300 develop abscesses that peak in size between 3-5 days after infection and then resolve between days 10-14. Consistent with human disease, a prior infection does not protect mice from subsequent challenge with the same organism (FIG. 4). However, mice immunized with MnDP-IRS+CFA showed decreased abscess formation upon challenge two weeks after the last immunization (FIG. 5a). Compared to mice immunized with IRS (either in phosphate buffered saline (PBS) or CFA), mice immunized with MnDP-IRS+CFA had significantly decreased abscess size (FIG. 5b) and skin MRSA bacterial burden (FIG. 5c). MnDP-IRS immunization in PBS without adjuvant showed a lesser degree of protection (FIG. 5b,c). To address if this vaccine-induced adaptive immune response was dependent on B cell antibody production or effector T cells, B cell-deficient (μMT) mice were immunized, or depleted CD4 T cells in wild-type mice were immunized prior to challenge. Loss of both B cells and CD4 T cells abrogated protection (FIG. 5d,e). However, loss of B cells alone or CD4 T cell depletion alone did not impact vaccine efficacy (FIG. 5d,e). Together, these data support that MnDP-IRS+CFA immunization elicits antibody- and CD4 T cell-mediated immunity that combine to protect against staphylococcal skin infection.

### Example 6

### Application of Reconstituted D. radiodurans Mn²⁺ Complexes for the Production of Irradiated Vaccines Against Venezuelan Equine Encephalitis Virus (VEEV)

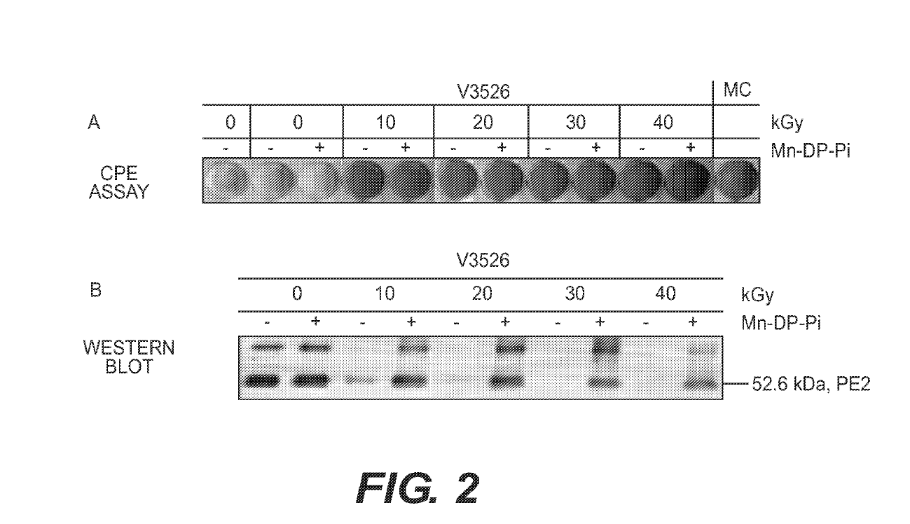

The use of Mn-DP-Pi for irradiating Venezuelan equine encephalitis virus (VEEV), an important human pathogen which causes encephalitis and for which a licensed protective killed vaccine does not exist, was examined. VEE viruses are a group of serologically related positive-stranded RNA viruses of the genus Alphavirus in the family Togaviridae. V3526 is a live attenuated strain derived from a full-length infectious done of VEEV, and previous studies examined its efficacy as an inactivated vaccine candidate. Infectivity and antibody binding capacity of V3526 irradiated to 0-40 kGy was tested in the presence or absence of Mn-DP-Pi. At 10 kGy in Pi buffer alone, loss of infectivity of V3526 (FIG. 2a) coincided with the initial marked decay in antibody binding capacity of PE2 (FIG. 2b), a surface glycoprotein of V3526; the reactivity of PE2 with the antibody was extinguished by 30 kGy. In stark contrast, PE2 of V3526 irradiated in Mn-DP-Pi retained its native antibody binding capacity at 40 kGy (FIG. 2b), but without increasing the survival of the virus (FIG. 2a). Thus, γ-radiation-induced killing was uncoupled from the destruction of the PE2 epitope in aqueous preparations of V3526 containing Mn-DP-Pi at doses extending to at least 40 kGy.

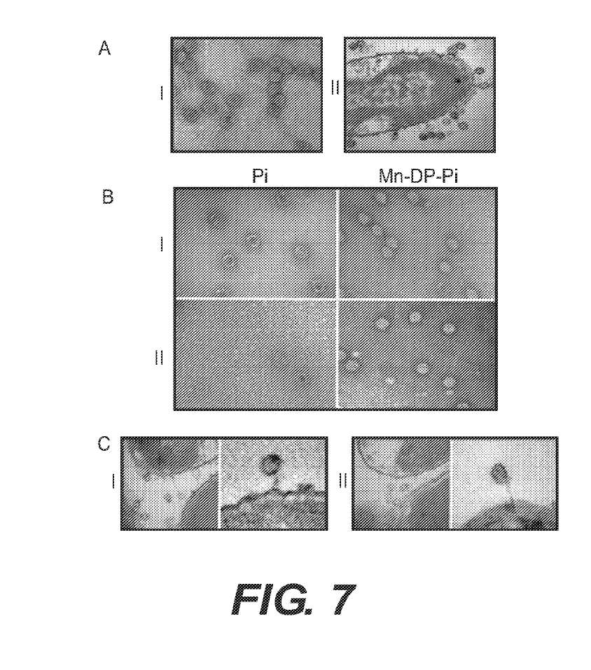

As an irradiated viral vaccine ideally would be based on a fully inactivated virus which displays undamaged surface protein conformations and superstructures, the distinctive morphological and adsorption properties of λ phage exposed to 40 kGy was examined (FIG. 7). Compared to non-irradiated λ phage (FIG. 7a), λ phage particles irradiated in Pi buffer alone lost their tails by 40 kGy (FIG. 7b,l) and the phage heads were rendered vulnerable to rupture (FIG. 7b, II). These structural changes tracked with dose-dependent increases in tail and head protein damage, and loss in immunogenicity (FIG. 1c,d). In contrast, normal head and tail morphologies were preserved in λ phage particles exposed to 40 kGy in Mn-DP-Pi (FIG. 7b, I and II). Further, λ phage exposed to 40 kGy in Mn-DP-Pi retained their ability to adsorb to E. coli (FIG. 7c, II). Thus, for λ phage particles exposed to supralethal doses of γ-rays (40 kGy) in Mn-DP-Pi, the structural integrity and binding capacities of the whole viruses were preserved (FIG. 7), and their immunogenic properties were indistinguishable from non-irradiated λ phage (FIG. 1d). As the genomes of λ phage (48.5 kb) exposed to 40 kGy in Mn-DP-Pi were obliterated (FIG. 1b), this approach presents itself as a novel route to preparing highly immunogenic lethally-irradiated intact viral vaccines.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1-12. (canceled)

13. A method of producing a vaccine directed against Venezuelan equine encephalitis virus (VEEV), the method comprising
a) culturing, harvesting, and/or suspending the VEEV in the presence of a radiation-protective composition, the composition comprising an antioxidant and at least one peptide of 25 amino acids or less, wherein the peptide comprises an amino acid sequence at least 80% identical to the amino acid sequence of SEQ ID NO:1,
irradiating the VEEV with a dose of radiation sufficient to render the VEEV replication-deficient.

14. The method of claim 13, wherein the radiation is selected from the group consisting of UV light, alpha radiation, beta radiation, gamma radiation, X-ray radiation and neutron radiation.

15. The method of claim 13, wherein the composition further comprises at least one nucleoside selected from the group consisting of adenosine, uridine, β-pseudouridine, inosine, and mixtures thereof.

16. The method of any of claim 15, wherein the at least one nucleoside is adenosine and uridine.

17. The method of any of claim 16, wherein the concentration of the at least one nucleoside is from about 1 mM to about 15 mM.

18. The method of claim 13, wherein the at least one antioxidant comprises manganese.

19. The method of claim 13, wherein concentration of the at least one antioxidant is from about 1 mM to about 12.5 mM of manganese.

20. The method of claim 19, wherein the at least one antioxidant is selected from group consisting of MnCl₂ and manganous phosphate.

21. The method of claim 13, wherein the composition further comprises at least one amino acid selected from the group consisting of alanine, valine and leucine.

22. The method of claim 13, wherein the composition further comprises phosphate.

23. The method of claim 13, wherein the composition further comprises an ultrafiltrate from D. radiodurans.

24. The method of claim 13, wherein the dose of radiation is at least about 20 kGy.

25. A vaccine comprising irradiated methicillin-resistant Staphylococcus aureus (MRSA), wherein the irradiated MRSA is antigenic when administered to a subject capable of generating an immune response.

26. A vaccine comprising irradiated Venezuelan equine encephalitis virus (VEEV), wherein the irradiated VEEV is antigenic when administered to a subject capable of generating an immune response.

27. A method of producing a vaccine directed against methicillin-resistant Staphylococcus aureus (MRSA), the method comprising
culturing, harvesting, and/or suspending the MRSA in the presence of a radiation-protective composition, the composition comprising an antioxidant and at least one peptide of 25 amino acids or less, wherein the peptide comprises an amino acid sequence at least 80% identical to the amino acid sequence of SEQ ID NO:1,
irradiating the MRSA with a dose of radiation sufficient to render the MRSA replication-deficient.

28. The method of claim 27, wherein the radiation is selected from the group consisting of UV light, alpha radiation, beta radiation, gamma radiation, X-ray radiation and neutron radiation.

29. The method of claim 27, wherein the composition further comprises at least one nucleoside selected from the group consisting of adenosine, uridine, β-pseudouridine, inosine, and mixtures thereof.

30. The method of any of claim 29, wherein the at least one nucleoside is adenosine and uridine.

31. The method of any of claim 30, wherein the concentration of the at least one nucleoside is from about 1 mM to about 15 mM.

32. The method of any of claim 27, wherein the at least one antioxidant comprises manganese.

33. The method of any of claim 27, wherein concentration of the at least one antioxidant is from about 1 mM to about 12.5 mM of manganese.

34. The method of claim 33, wherein the at least one antioxidant is selected from group consisting of MnCl₂ and manganous phosphate.

35. The method of any of claim 27, wherein the composition further comprises at least one amino acid selected from the group consisting of alanine, valine and leucine.

36. The method of any of claim 27, wherein the composition further comprises phosphate.

37. The method of any of claim 27, wherein the composition further comprises an ultrafiltrate from D. radiodurans.

38. The method of any of claim 27, wherein the dose of radiation is at least about 20 kGy.
